(19) United States
(12) Patent Application Publication
(10) Pub. No.: US 2024/0071605 A1
(43) Pub. Date: Feb. 29, 2024

(54) INTELLIGENT ASSISTANT FOR THE COMMUNICATION OF MEDICAL INFORMATION

(71) Applicant: AT&T Intellectual Property I, L.P., Atlanta, GA (US)

(72) Inventors: Nigel Bradley, Canton, GA (US); Eric Zavesky, Austin, TX (US); James Pratt, Round Rock, TX (US); Ari Craine, Marietta, GA (US); Robert Koch, Peachtree Corners, GA (US)

(21) Appl. No.: 17/822,583

(22) Filed: Aug. 26, 2022

(51) Int. Cl. G16H 40/20; G16H 80/00; G06V 40/10

(52) U.S. Cl. CPC G16H 40/20; G16H 80/00; G06V 40/10; G06V 2201/03

(57) ABSTRACT

The disclosed technology is directed towards presenting a medical practitioner with information that assists the practitioner in communicating with a given patient. Baseline data describing a medical patient is obtained, such as from one or more previous visits. The baseline data can be compared with current data, e.g., sensed by patient biosensors and/or discerned from a current video of the patient during the visit. Historical and/or known healthy data can be used as baseline data. Interaction data representing an interaction (e.g., a conversation during a current visit) between the medical practitioner and the medical patient is determined. Based on the baseline data and the interaction data, during the interaction between the medical practitioner and the medical patient the medical practitioner can be presented with coaching data to assist the medical practitioner in communicating with the patient.

### TECHNICAL FIELD

The subject application relates to the communication of medical information, such as via augmented reality applications, and related embodiments.

### BACKGROUND

Medical practitioners often have difficulty in interacting with patients. In part because different patients receive and react to different medical information in different ways. Some patients, for example, are highly informed with respect to medical knowledge, at least regarding their own conditions, whereas other patients are not particularly informed. Other times a patient may be undergoing different circumstances during different interactions with medical practitioners; as some examples, a patient may be feeling differently relative to a previous visit, a different medical practitioner may be interacting with the patient, and so on.

### BRIEF DESCRIPTION OF THE DRAWINGS

Non-limiting and non-exhaustive embodiments of the subject disclosure are described with reference to the following figures, wherein like reference numerals refer to like parts throughout the various views unless otherwise specified.

FIG. 1 is a block diagram of an example system/architecture for processing and communicating medical information between a patient and medical practitioner, in accordance with various aspects and embodiments of the subject disclosure.

FIG. 2 is a block diagram of an example system/architecture, including using patient profile data for processing and communicating medical information, in accordance with various aspects and embodiments of the subject disclosure.

FIG. 3 is an example representation of using augmented reality with respect to presenting patient profile data to medical practitioners, in accordance with various aspects and embodiments of the subject disclosure.

FIGS. 4 and 5 are example representations related to presenting patient condition data to medical practitioners, in accordance with various aspects and embodiments of the subject disclosure.

FIG. 6 is an example representation of summarized medical data presented in audio form to a medical practitioner, in accordance with various aspects and embodiments of the subject disclosure.

FIG. 7 is an example representation of presenting patient condition data, relative to one or more previous visits, to a medical practitioner, in accordance with various aspects and embodiments of the subject disclosure.

FIG. 8 is an example representation of presenting coaching data to a medical practitioner based on an analysis of patient data, in accordance with various aspects and embodiments of the subject disclosure.

FIG. 9 is an example representation of presenting coaching data to a medical practitioner in an audio form, in accordance with various aspects and embodiments of the subject disclosure.

FIG. 10 is a flow diagram representing example operations related to presenting coaching assistance data to a medical practitioner during an interaction with medical patient, based on patient baseline data, in accordance with various aspects and embodiments of the subject disclosure.

FIG. 11 is a flow diagram representing example operations related to presenting coaching assistance data to a medical practitioner during an interaction with medical patient, based on first and second patient baseline data, in accordance with various aspects and embodiments of the subject disclosure.

FIG. 12 is a flow diagram representing example operations related to presenting coaching assistance data to a second medical practitioner based on health-related state data obtained based on a visit with a first medical practitioner, in accordance with various aspects and embodiments of the subject disclosure.

FIG. 13 illustrates an example block diagram of an example mobile handset operable to engage in a system architecture that facilitates wireless communications according to one or more embodiments described herein.

FIG. 14 illustrates an example block diagram of an example computer/machine system operable to engage in a system architecture that facilitates wireless communications according to one or more embodiments described herein.

### DETAILED DESCRIPTION

The technology described herein is generally directed towards providing medical practitioners with an improved way to understand how to better interact with patients. As different patients receive and react to different information in different ways, the technology described herein provides a system that facilitates such improved interactions.

In one aspect, medical practitioners, in the course of conducting a patient visit, are presented with access to data (e.g., via virtual reality) that enable a more effective and efficient communication of information between a medical practitioner, including in ways that generally do not interrupt the flow of communication between them and the patient. This data can help a practitioner better remember the particulars, including needs, of a specific patient.

The practitioner may be enabled to better communicate information and receive information from the patient such that a more beneficial effect is achieved. The technology described herein can use per-patient data and thereby take into account each specific patient's needs, the patient's history and other factors, including the patient's current condition data. The technology can further coach the practitioner so as to adjust how the practitioner conveys information to the patient, e.g., in a way that such information is best received and comprehended by that patient.

As used in this disclosure, in some embodiments, the terms “component,” “system” and the like are intended to refer to, or include, a computer-related entity or an entity related to an operational apparatus with one or more specific functionalities, wherein the entity can be either hardware, a combination of hardware and software, software, or software in execution. As an example, a component may be, but is not limited to being, a process running on a processor, a processor, an object, an executable, a thread of execution, computer-executable instructions, a program, and/or a computer. By way of illustration and not limitation, both an application running on a server and the server can be a component.

One or more components may reside within a process and/or thread of execution and a component may be localized on one computer and/or distributed between two or more computers. In addition, these components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software application or firmware application executed by a processor, wherein the processor can be internal or external to the apparatus and executes at least a part of the software or firmware application. As yet another example, a component can be an apparatus that provides specific functionality through electronic components without mechanical parts, the electronic components can include a processor therein to execute software or firmware that confers at least in part the functionality of the electronic components. While various components have been illustrated as separate components, it will be appreciated that multiple components can be implemented as a single component, or a single component can be implemented as multiple components, without departing from example embodiments.

Further, the various embodiments can be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer program accessible from any computer-readable (or machine-readable) device or computer-readable (or machine-readable) storage/communications media. For example, computer readable storage media can include, but are not limited to, magnetic storage devices (e.g., hard disk, floppy disk, magnetic strips), optical disks (e.g., compact disk (CD), digital versatile disk (DVD)), smart cards, and flash memory devices (e.g., card, stick, key drive). Of course, those skilled in the art will recognize many modifications can be made to this configuration without departing from the scope or spirit of the various embodiments.

Moreover, terms such as “mobile device equipment,” “mobile station,” “mobile,” subscriber station,” “access terminal,” “terminal,” “handset,” “communication device,” “mobile device” (and/or terms representing similar terminology) can refer to a wireless device utilized by a subscriber or mobile device of a wireless communication service to receive or convey data, control, voice, video, sound, gaming or substantially any data-stream or signaling-stream. The foregoing terms are utilized interchangeably herein and with reference to the related drawings. Likewise, the terms “access point (AP),” “Base Station (BS),” BS transceiver, BS device, cell site, cell site device, “gNode B (gNB),” “evolved Node B (eNode B),” “home Node B (HNB)” and the like, can be utilized interchangeably in the application, and can refer to a wireless network component or appliance that transmits and/or receives data, control, voice, video, sound, gaming or substantially any data-stream or signaling-stream from one or more subscriber stations. Data and signaling streams can be packetized or frame-based flows.

Furthermore, the terms “user equipment,” “device,” “communication device,” “mobile device,” “subscriber,” “customer entity,” “consumer,” “customer entity,” “entity” and the like may be employed interchangeably throughout, unless context warrants particular distinctions among the terms. It should be appreciated that such terms can refer to human entities or automated components supported through artificial intelligence (e.g., a capacity to make inference based on complex mathematical formalisms), which can provide simulated vision, sound recognition and so forth. Olfactory output as well as taste output and/or tactile output can also be part of a promotional presentation as described herein.

Embodiments described herein can be exploited in substantially any wireless communication technology, including, but not limited to, wireless fidelity (Wi-Fi), global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra mobile broadband (UMB), high speed packet access (HSPA), Z-Wave, Zigbee and other 802.11 wireless technologies and/or legacy telecommunication technologies.

One or more embodiments are now described with reference to the drawings, wherein like reference numerals are used to refer to like elements throughout. In the following description, for purposes of explanation, numerous specific details are set forth in order to provide a thorough understanding of the various embodiments. It is evident, however, that the various embodiments can be practiced without these specific details (and without applying to any particular networked environment or standard).

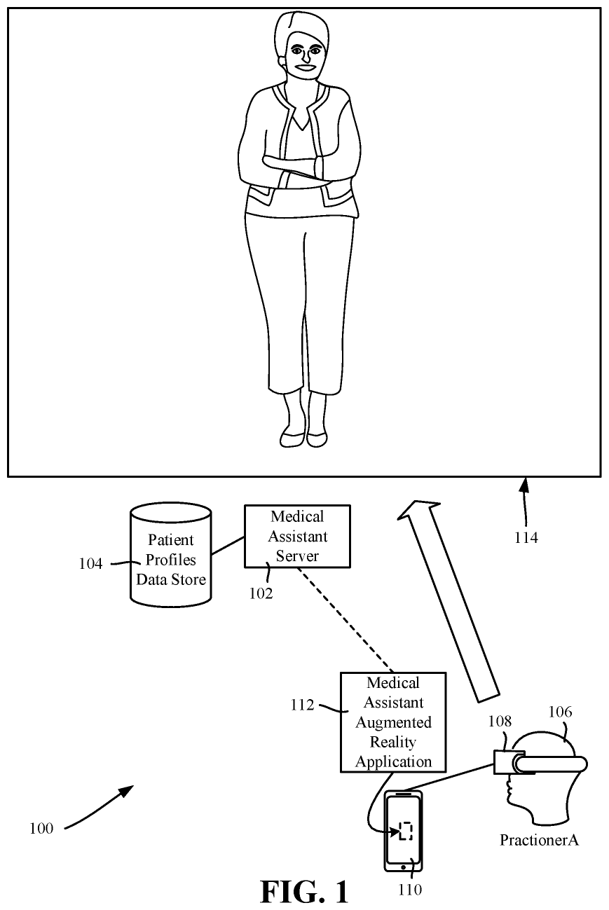

FIG. 1 shows an example system/architecture 100 including a medical assistant server 102 coupled to a patient profiles data store 104. One or more practitioners (one such practitioner 106, PractitionerA) is depicted in the example of FIG. 1) may have access to augmented reality viewing devices. In FIG. 1, a viewing device 108 is shown as being worn by the PractitionerA 106, e.g., smart glasses or other viewing headset, which can be coupled to a smartphone or other smart device 110. The viewing device 108 may include processing and memory capability, such as for running programs, logic and the like. If coupled to a smart device 110, at least some of the processing and memory of the smart device 110 can be leveraged. The augmented reality device(s) 108 may have an integrated audio speaker such as earbuds, or be coupled to such a device. Alternatively or in addition to a separate viewing device, the smart device 110 may serve as the augmented reality viewing device and/or audio output device.

The augmented reality viewing device, smart device and/or earbuds may be in communication with (which may be by running) a medical assistant augmented reality application program 112, shown in this example as being run incorporated into the smart device 110 (but alternatively can be incorporated into the viewing device 108). The medical assistant augmented reality application program 112 is in communication with the medical assistant server 102, which has access to patient profile data via the patient profiles data store 104.

In general, the medical assistant augmented reality application program 112 is used to present augmented reality information to one or more practitioners in visual and/or audio form; in FIG. 1, a visual representation 114 (not yet augmented) of a medical patient is shown as viewed from the perspective of the PractitionerA 106.

The patient profile data that is maintained and, when appropriate presented, can be collected from any saved patient profile information, can be generated by analysis by the medical assistant server 102, and/or can be generated based on one or more sensors that are sensing medical conditions for the patient. Such sensors can include, but are not limited to, biosensors worn by the patient, cameras that view the patient, microphones that listen to the patient, devices temporarily contacted by the patient (e.g., an EKG coupled to a smartphone or the like) and so forth. Practitioner input, whether via a live visit or virtual visit (e.g., via camera and microphone) can also supplement the patient profile data. Patient input as well can be used; e.g., the patient may complete a survey of conditions, family history of medical issues, known allergies, and so forth.

During a first visit, with PractitionerA 106 for example, a baseline set of data may be established for a medical patient. The baseline data may be stored in the patient's profile data in the patient profile data store 104. This baseline data can include vital signs, such as heart rate, respiration, pulse oxygen level, and other. The baseline data can also include data that is obtained from the medical assistant server 102 based on an analysis of video and/or audio obtained by and presented by the medical assistant augmented reality application 112.

To this end, the augmented reality viewer, and/or separate camera(s), may have video capture capabilities and may be used to send video of the patient to the medical assistant server 102 for analysis. The medical assistant server 102 may analyze the video and generate some (or all) of the patient's baseline data representing a baseline state for conditions such as the patient's perspiration level, skin pallor, body posture, facial expression, and other factors. Audio may also be analyzed, e.g., voice strength, any shakiness, slurred speech and so on can be evaluated and used as part of the baseline data for a patient.

Note that because augmented reality viewers, cameras, lighting and other factors sense imagery differently among different devices and/or at different times, it may be useful to calibrate the video sensors in some way. For example, a known color chart such as a wheel or color bars may be behind the patient or held by the patient during part of a visit. This is straightforward during an in-person visit, for example, but can be sent as part of a “kit” for in-home virtual visits. Any corrections such as to sensed skin pallor can be applied based on the known color data accompanying the images. Similarly, microphone volume can be calibrated. In this way, colors and/or sounds sensed during different visits can be more accurately compared.

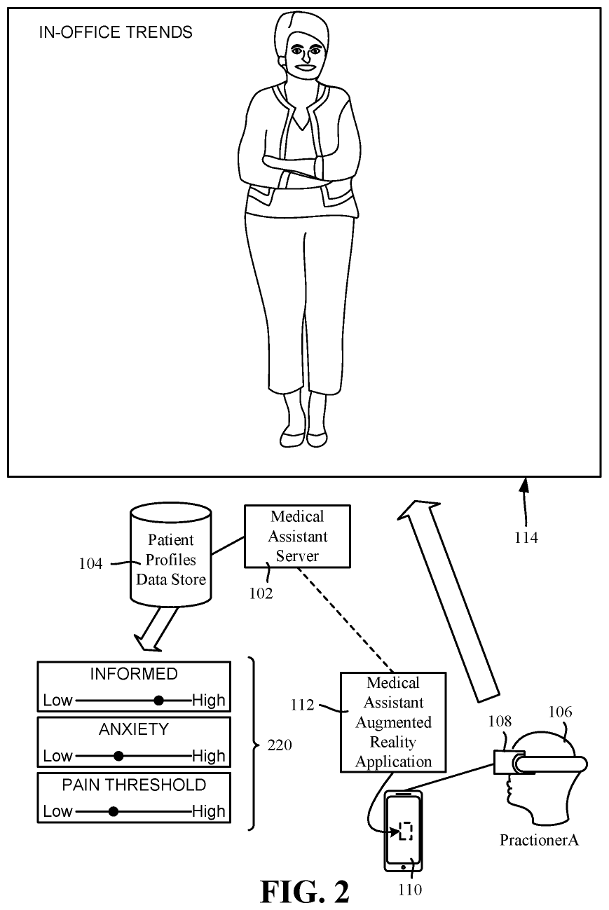

In addition to sensing and determining baseline data, during the first visit with a practitioner (the PractitionerA 106 in this example), conversational interaction between the practitioner and the patient may be captured (audio and video) and sent to the medical assistant server 102 for analysis with respect to the patient's personal factors. The medical assistant server 102 may use artificial intelligence and/or machine learned techniques to analyze the context of audio and video of the conversation to estimate a number of such factors about the patient, which may include, but are not limited to, how informed or inquisitive she is, how anxious she is, what her pain threshold typically is, and/or other such factors. These estimations may be made based on the captured verbal information (possibly via sign language or through an interpreter) such as content, context, terminology and language used, visual cues such as facial expressions and gestures, voice intonation, and other audio and visual analysis. As represented in FIG. 2, these data 220 may be stored as scores for patient profile factors such as informed level, anxiety level and pain threshold level. These data can assist in coaching the practitioner's communication with the patient, e.g., in choosing terms, emphasizing certain concepts, slowing down or speeding up speech, tone of speech, and so forth.

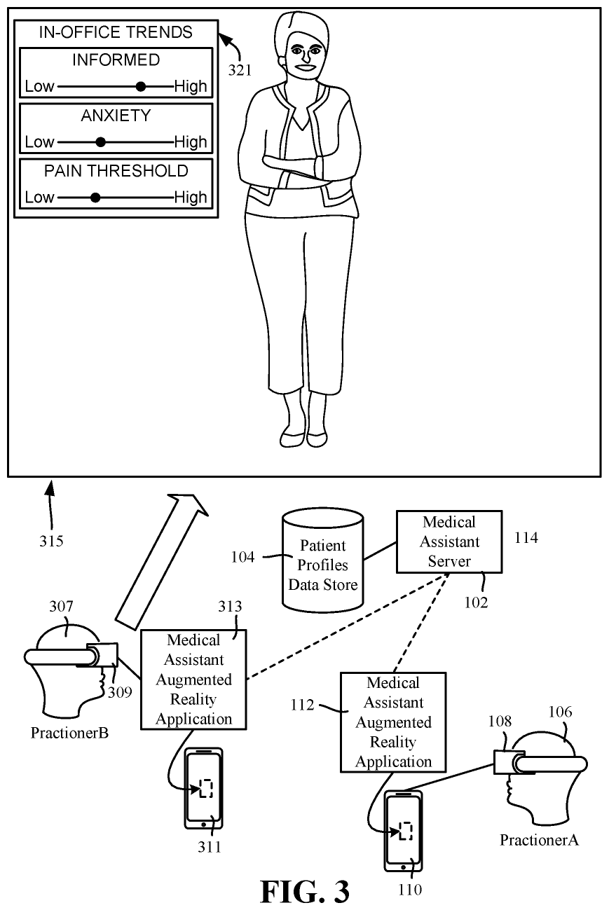

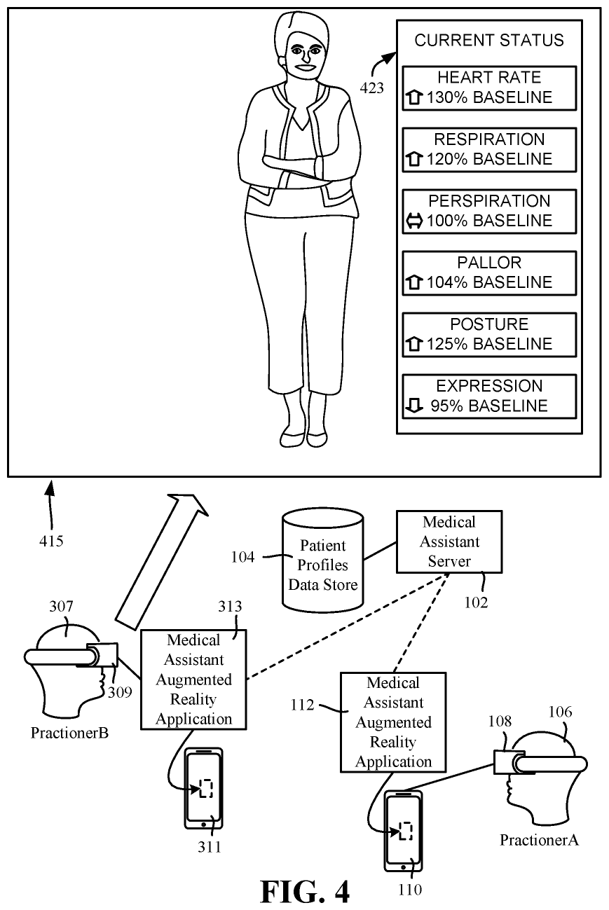

The patient may encounter a subsequent practitioner visit. The subsequent visit may be with the same PractitionerA 106, or may be with a different practitioner PractitionerB 307, as shown in FIG. 3. In the example of FIG. 3, the PractitionerB 307 has viewing device 309, a smart device 311 and is communicating with the medical assistant server 102 via another instance of the medical assistant augmented reality application, labeled 313.

In conducting and/or preparing for the subsequent patient visit, the practitioner may be presented with an augmented reality presentation 315 of the patient, and data related to the patient, e.g., augmented reality trend data 321. The patient data from the patient profile data store 104 may be accessed by using the patient's face as a facial recognition tool to access their data. If the patient is currently visible to the system, facial identification, which may be stored in the patient profile, may be as the key to the other patient profile data. Alternatively, other mechanisms may be used to access the patient's profile data. Note that while the augmented reality presentation 315 is shown from the perspective of the PractitionerB 307, it is possible that the PractitionerA 106 is also receiving a similar (or the same) view. For example, a specialist may be overseeing a visit; an intern may be being supervised by a more experienced doctor, and so on. It is also feasible to record a visit for subsequently re-experiencing the visit, possibly by the same practitioner or a different one (e.g., send this recording to the specialist).

The trends data 321 for a medical patient may be presented as coaching assistance data in a number of ways, such as to visibly help coach the practitioner. As shown in the example, such data may be presented as augmented reality data that shows the practitioner a scoring view of the data. Alternatively, this data may be presented in audio format by the server sending the data as to the application 313 for a presentation as audio output, e.g., via the earbuds. Among other benefits, the presentation of this data 321 enables the practitioner to adjust their style and content of delivery of information to the patient so as to best meet the patient's needs. Note that in the non-limiting example of FIG. 3 the term “in-office” trends data 321 somewhat suggests that the patient is in an office such as a doctor's office or clinic, the patient can be in any suitable location, e.g., at home, with the visit being a virtual “in-office” visit.

Based on the content and context of the subsequent practitioner visit, the subsequent visit conversation may be sent to the medical assistance server 102 for further analysis. Based on this analysis, the in-office trends data for the patient may be updated. Because in-office trends data may vary for the patient from practitioner to practitioner, this data may be optionally stored for each practitioner separately for the patient. In any event, the in-office trends data may be updated after each visit by this conversational audio and video analysis. Note that baseline data also can be updated and/or maintained separately over a set of visits; for example, a patient regularly obtaining annual checkups will age over time, and what a patient had as baseline data at age twenty-five likely will not be as relevant at age forty compared to more recently determined (or updated) baseline data.

Note that the trends data and/or the baseline data can be presented on a per-visit presentation, or a composite of multiple visits. A practitioner can interact, for example, to see or hear the data from the most recent previous visit, from the second most recent previous visit, from a year ago, from a month ago, the most recent n visits, all visits, and so on.

Also during a subsequent visit, video from the practitioner's device and data from biosensors and other sensors (e.g., cameras and microphones) that are monitoring the patient may be sent to the medical assistant server 102 for analysis. Biosensor data may include, but is not limited to, heart rate, respiration rate, pulse oxygen level, EKG data, and data from other sensors. The medical assistant server 102 may compare the current real-time data readings to those from the baseline patient profile and present an augmented reality presentation of the current status data 423 shown in the modified augmented reality view 415, highlighting any divergence from what is deemed normally healthy baseline data, e.g., overall, or for a person of a similar age group, sex or other grouping (e.g., with a similar disease or disability). A full-body view of the patient may also collect video that the server may use to compare against baseline data to determine changes in body posture, gait or other baseline healthy data values. These data can help the medical assistant server 102 determine the patient's current and/or relative state to help coach the medical practitioner, which the medical practitioner may not discern. For example, if heart rate is up, perspiration is high, breathing is fast and shallow, etc.; the medical assistant server 102 may determine that the medical patient appears unusually nervous or otherwise stressed and provide this data as a type of coaching assistance data to the practitioner.

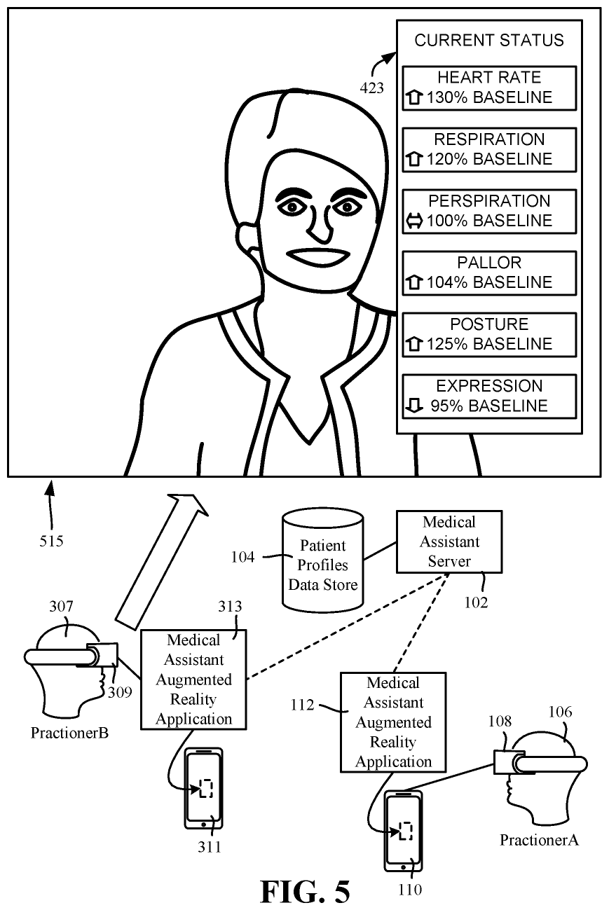

As represented in the example of FIG. 5, a closer (e.g., zoomed-in) view 515 of the patient may be used to more accurately collect data that may be used to determine other current status factors of the patient. For example, perspiration levels may be detected based on an analysis of the video. Also, an analysis of color levels of the video may be used to determine any pallor in the patient's skin that may indicate a divergence from a baseline normal color. Further, an analysis of the patient's facial expression may be used to determine a higher level of worry, fear, or other negative emotional condition. Any of these determined current status factors may help coach the practitioner, e.g., in making a diagnosis or evaluating a known condition.

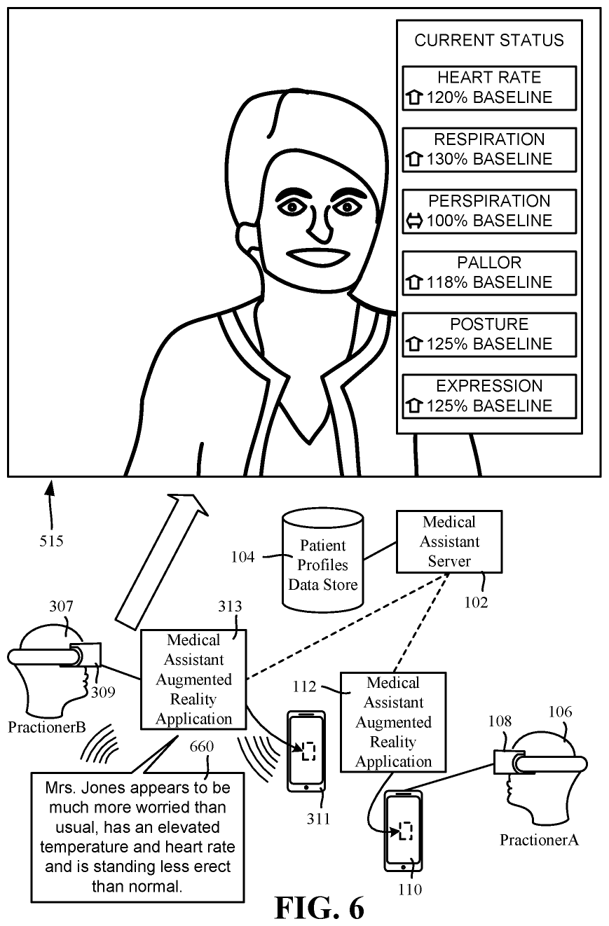

As set forth herein and as represented in FIG. 6, the data may be presented optionally in an audio form (block 660) to the practitioner. In this matter, the medical assistant AR app may summarize the data in a more condensed way so as to minimize distraction from a conversation with a patient. Such audio data can be considered one form of coaching data presented to the practitioner.

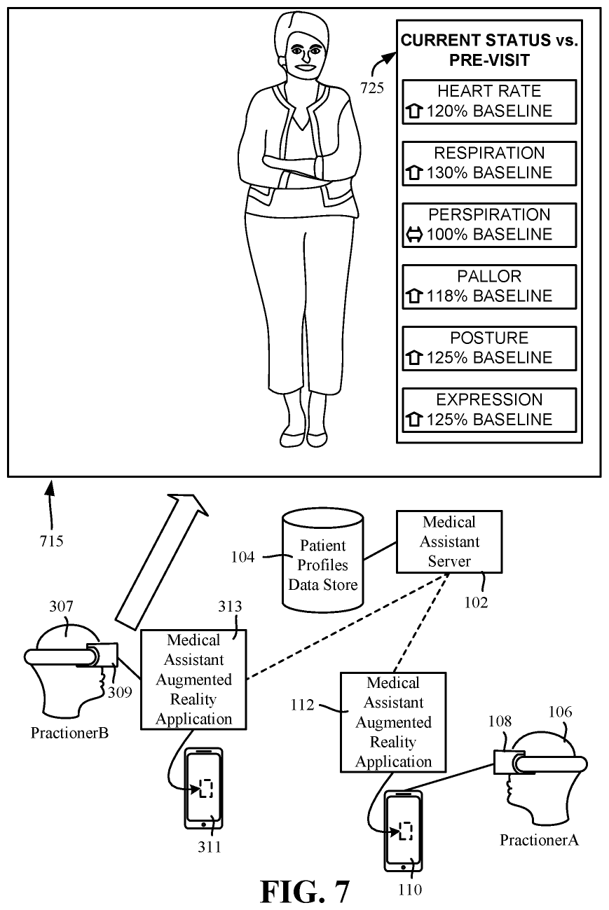

At times, a patient's data may vary based purely on the anxiety level associated with the practitioner visit itself. To help determine if this is a factor, the patient's data may be compared over a short time frame, such as on the same day, e.g., before and during their visit. In this embodiment generally shown in the example view 715 of FIG. 7, the current status data 725 may be presented versus pre-visit data from one (or more) previous visits. Sensors may be used to gather data from the patient before their visit and may be stored in the patient profile for the patient. This pre-visit data may be collected using biosensors that are worn by the patient or other sensors such as video that the patient creates of themselves and records and sends to the patient profile before the visit. Note that this can also be for previous visit(s) with a different practitioner; for example, if interaction with the medical patient is normally by PractitionerA 106, but for this visit is with PractitionerB 307, it is likely that the different practitioner will influence the patient's anxiety level.

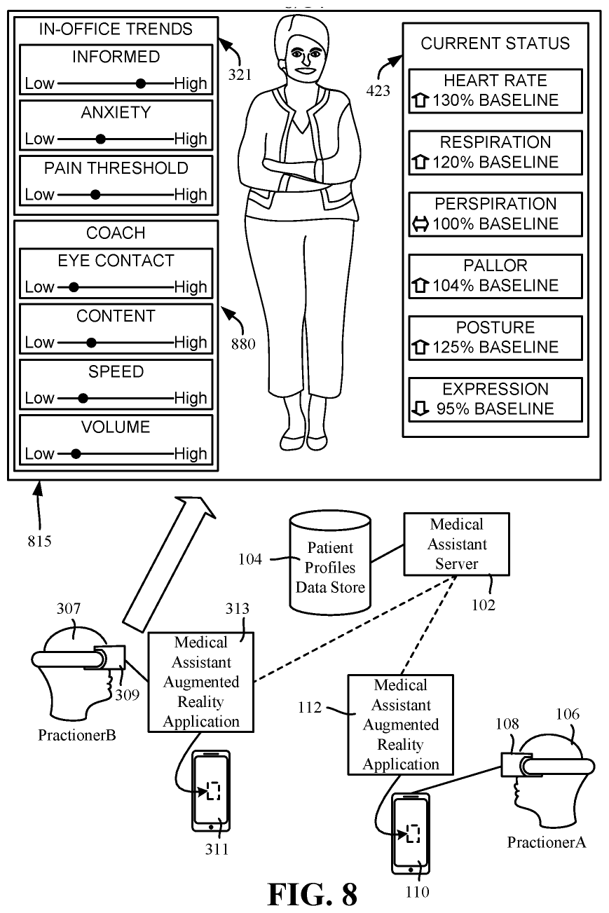

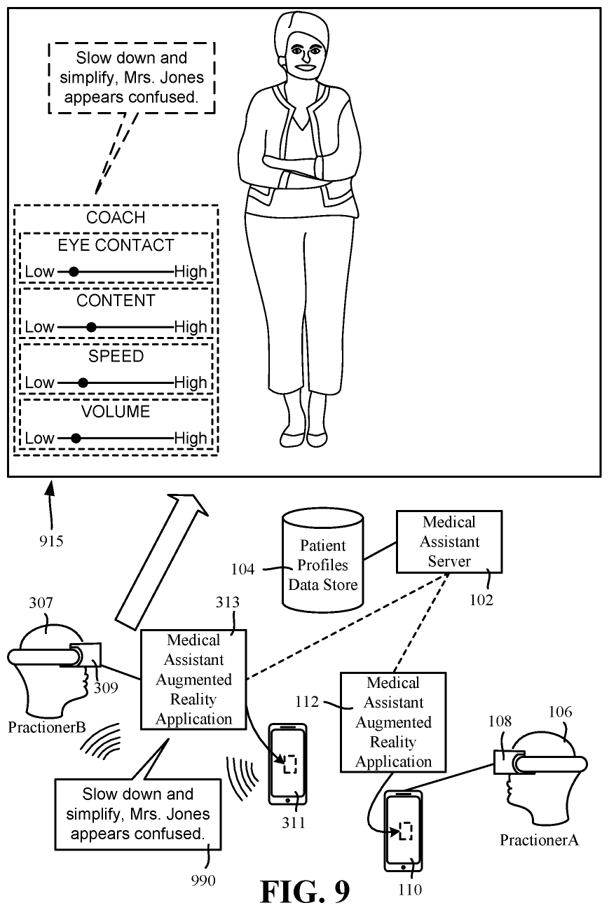

Turning to further practitioner coaching, data describing the conversation between the practitioner and the patient may also be sent to the medical assistant server for real-time analysis and feedback to the practitioner, e.g., the PractitionerB 307. For example, as shown in the augmented reality view 815 of FIG. 8, the applicator 313 may detect and record the practitioner's eye gaze data, e.g., focus, direction of eye contact such as forwards towards the patient, for instance, as opposed to downwards towards papers or a device. The level of eye contact (and/or other eye gaze data) that is presented may be updated and shown throughout the visit, e.g., via the augmented reality coach representation 880. Also, an analysis of the data describing the conversation may yield feedback that may be presented as additional coaching assistance data to the practitioner regarding the complexity of the content of information that they speak to the patient, as well as the speed and volume with which they are speaking. Other detected practitioner data (not explicitly shown in FIG. 9) can be used in determining how and with what information to coach the practitioner, such as sensed practitioner gestures, practitioner sensed excitement, agitation or apprehension, how often the practitioner is repeating the same content, and so on.

These levels that can help coach the practitioner may, in part, be determined based on reactions of/responses from the patient. The reactions/responses may be a change in the patient's expression, posture, or gestures (e.g., hand to ear), for example, or other current status factors, as well as based on the content of the dialogue of the patient.

As described herein, any coaching assistance data may be alternatively presented in an audible format. FIG. 9 shows an example in which not only is the coaching data audible, but is used to determine and present a course of action for the practitioner based on an analysis by the medical assistant server 102 of the above described factors, e.g., eye gaze data, and speech content, speed and volume, along with an analysis of the patient's reactions/responses. Such a coaching conclusion/summarization (block 990) can alternatively (or in addition to audibly) be presented to the practitioner visibly (represented in the augmented reality view 915 via the dashed blocks in FIG. 9).

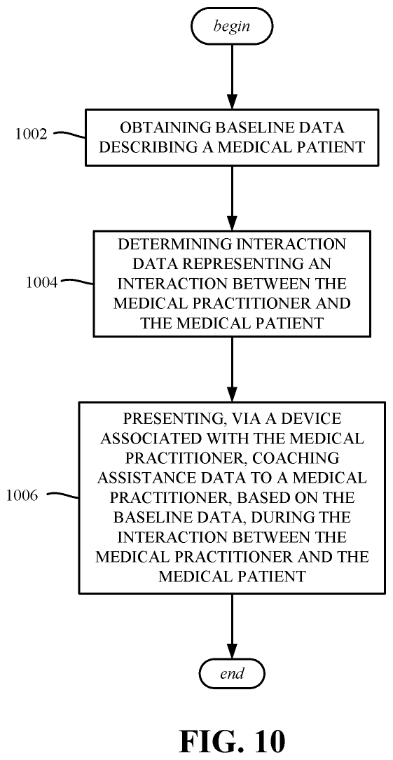

One or more example aspects are represented in FIG. 10, and can correspond to a system, including a processor, and a memory that stores executable instructions that, when executed by the processor, facilitate performance of operations. Example operation 1002 represents obtaining baseline data describing a medical patient. Example operation 1004 represents determining interaction data representing an interaction between the medical practitioner and the medical patient. Example operation 1006 represents presenting, via a device associated with the medical practitioner, coaching assistance data to a medical practitioner, based on the baseline data, during the interaction between the medical practitioner and the medical patient.

The baseline data can include first baseline data, and further operations can include updating the first baseline data into second baseline data based on the interaction data.

The baseline data describing the medical patient can include information determined from analysis of medical patient data collected before the interaction between the medical practitioner and the medical patient.

The baseline data describing the medical patient can include video data.

The baseline data describing the medical patient can include biometric data.

Presenting the coaching assistance data can include outputting, via the device, augmented reality data in association with image data representing the medical patient during the interaction between the medical practitioner and the medical patient.

Presenting the coaching assistance data can include outputting, via the device, audio data during the interaction between the medical practitioner and the medical patient.

Presenting the coaching assistance data can include presenting, via the device, medical patient trend data during the interaction between the medical practitioner and the medical patient.

Presenting the coaching assistance data can include presenting, via the device, current status data relative to the baseline data during the interaction between the medical practitioner and the medical patient.

Presenting the coaching assistance data can include presenting, via the device, current status data relative to the baseline data, the current status data can include at least one of: heart rate data, respiration data, perspiration data, pallor data, posture data, or expression data.

Obtaining the baseline data describing the medical patient can include obtaining information representative of at least one of: medical patient skin color level data, medical patient body posture, medical patient gait, medical patient facial expression, medical patient perspiration level, or medical patient eye gaze data.

Obtaining the baseline data describing the medical patient can include obtaining image-based information representative of medical patient skin color level data, and adjusting the image-based information based on predetermined color calibration information obtained in conjunction with the obtaining of the image-based information.

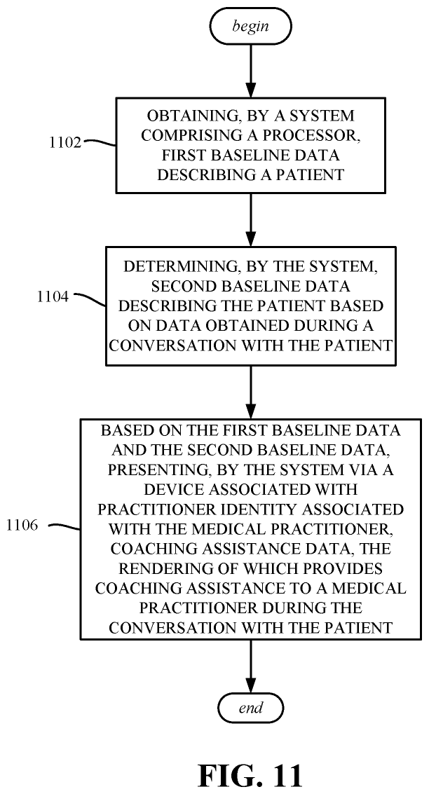

One or more example aspects are represented in FIG. 11, and, for example, can correspond to operations, such as of a method. Example operation 1102 represents obtaining, by a system comprising a processor, first baseline data describing a patient. Example operation 1104 represents determining, by the system, second baseline data describing the patient based on data obtained during a conversation with the patient. Example operation 1106 represents, based on the first baseline data and the second baseline data, presenting, by the system via a device associated with practitioner identity associated with the medical practitioner, coaching assistance data, the rendering of which provides coaching assistance to a medical practitioner during the conversation with the patient.

Obtaining the first baseline data can include obtaining the first baseline data based on a prior conversation with the patient.

Obtaining the first baseline data can include obtaining at least one of: patient biometric sensor data, or patient image data.

Presenting the coaching assistance data to the medical practitioner can include outputting at least one of: augmented reality data, or audio data.

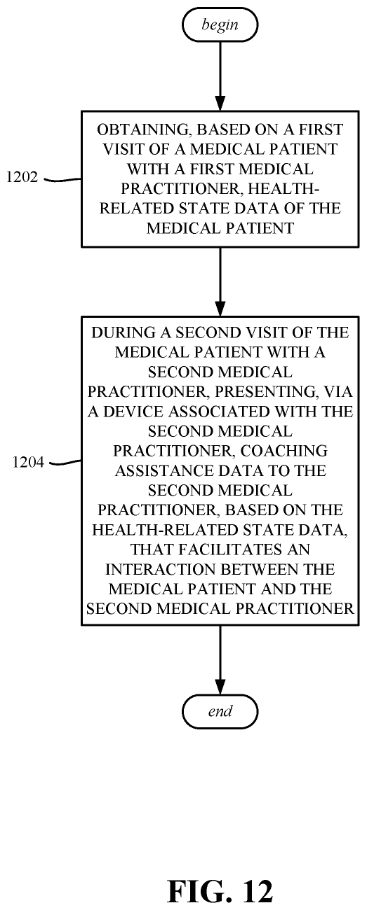

One or more aspects are represented in FIG. 12, such as implemented in a machine-readable medium, including executable instructions that, when executed by a processor, facilitate performance of operations. Example operation 1202 represents obtaining, based on a first visit of a medical patient with a first medical practitioner, health-related state data of the medical patient. Example operation 1204 represents, during a second visit of the medical patient with a second medical practitioner, presenting, via a device associated with the second medical practitioner, coaching assistance data to the second medical practitioner, based on the health-related state data, that facilitates an interaction between the medical patient and the second medical practitioner.

Presenting the coaching assistance data can include presenting, via the device, trend data representative of at least one of: an informed state of the medical patient, an anxiety state of the medical patient, or a pain threshold state of the medical patient.

Further operations can include updating the health-related state data of the medical patient into updated health-related state data based on information obtained via the second visit of the medical patient with the second medical practitioner.

The device can be a first device, the coaching assistance data can be first coaching assistance data, and further operations can include, during a third visit of the medical patient with a third medical practitioner, presenting, via a second device associated with the third medical practitioner, second coaching assistance data to the third medical practitioner, based on the updated health-related state data.

As can be seen, the technology described herein facilitates more effective and efficient communication of information between a medical practitioner and patient. This is accomplished in part by readily providing the medical practitioner with access to data in the course of conducting a patient visit. By providing the practitioner access to this data in a timely and meaningful way, without interrupting the flow of communication between them and the patient, the practitioner is likely better able to communicate information to and receive information from the patient such that a more beneficial effect is achieved. The technology described herein takes into account each specific patient's needs and may be informed by the patient's history with that practitioner, or with other practitioners, or other historical trends. The technology described herein also provides for coaching feedback in real time to the practitioner, whereby he or she can adjust conveying of information to the patient in a way that it is best received by that patient.

Turning to aspects in general, a wireless communication system can employ various cellular systems, technologies, and modulation schemes to facilitate wireless radio communications between devices (e.g., a UE and the network equipment). While example embodiments might be described for 5G new radio (NR) systems, the embodiments can be applicable to any radio access technology (RAT) or multi-RAT system where the UE operates using multiple carriers e.g. LTE FDD/TDD, GSM/GERAN, CDMA2000 etc. For example, the system can operate in accordance with global system for mobile communications (GSM), universal mobile telecommunications service (UMTS), long term evolution (LTE), LTE frequency division duplexing (LTE FDD, LTE time division duplexing (TDD), high speed packet access (HSPA), code division multiple access (CDMA), wideband CDMA (WCMDA), CDMA2000, time division multiple access (TDMA), frequency division multiple access (FDMA), multi-carrier code division multiple access (MC-CDMA), single-carrier code division multiple access (SC-CDMA), single-carrier FDMA (SC-FDMA), orthogonal frequency division multiplexing (OFDM), discrete Fourier transform spread OFDM (DFT-spread OFDM) single carrier FDMA (SC-FDMA), Filter bank based multi-carrier (FBMC), zero tail DFT-spread-OFDM (ZT DFT-s-OFDM), generalized frequency division multiplexing (GFDM), fixed mobile convergence (FMC), universal fixed mobile convergence (UFMC), unique word OFDM (UW-OFDM), unique word DFT-spread OFDM (UW DFT-Spread-OFDM), cyclic prefix OFDM CP-OFDM, resource-block-filtered OFDM, Wi Fi, WLAN, WiMax, and the like. However, various features and functionalities of system are particularly described wherein the devices (e.g., the UEs and the network equipment) of the system are configured to communicate wireless signals using one or more multi carrier modulation schemes, wherein data symbols can be transmitted simultaneously over multiple frequency subcarriers (e.g., OFDM, CP-OFDM, DFT-spread OFDM, UFMC, FMBC, etc.). The embodiments are applicable to single carrier as well as to multicarrier (MC) or carrier aggregation (CA) operation of the UE. The term carrier aggregation (CA) is also called (e.g. interchangeably called) “multi-carrier system”, “multi-cell operation”, “multi-carrier operation”, “multi-carrier” transmission and/or reception. Note that some embodiments are also applicable for Multi RAB (radio bearers) on some carriers (that is data plus speech is simultaneously scheduled).

In various embodiments, the system can be configured to provide and employ 5G wireless networking features and functionalities. With 5G networks that may use waveforms that split the bandwidth into several sub-bands, different types of services can be accommodated in different sub-bands with the most suitable waveform and numerology, leading to improved spectrum utilization for 5G networks. Notwithstanding, in the mmWave spectrum, the millimeter waves have shorter wavelengths relative to other communications waves, whereby mmWave signals can experience severe path loss, penetration loss, and fading. However, the shorter wavelength at mmWave frequencies also allows more antennas to be packed in the same physical dimension, which allows for large-scale spatial multiplexing and highly directional beamforming.

Performance can be improved if both the transmitter and the receiver are equipped with multiple antennas. Multi-antenna techniques can significantly increase the data rates and reliability of a wireless communication system. The use of multiple input multiple output (MIMO) techniques, which was introduced in the third-generation partnership project (3GPP) and has been in use (including with LTE), is a multi-antenna technique that can improve the spectral efficiency of transmissions, thereby significantly boosting the overall data carrying capacity of wireless systems. The use of multiple-input multiple-output (MIMO) techniques can improve mmWave communications; MIMO can be used for achieving diversity gain, spatial multiplexing gain and beamforming gain.

Note that using multi-antennas does not always mean that MIMO is being used. For example, a configuration can have two downlink antennas, and these two antennas can be used in various ways. In addition to using the antennas in a 2×2 MIMO scheme, the two antennas can also be used in a diversity configuration rather than MIMO configuration. Even with multiple antennas, a particular scheme might only use one of the antennas (e.g., LTE specification's transmission mode 1, which uses a single transmission antenna and a single receive antenna). Or, only one antenna can be used, with various different multiplexing, precoding methods etc.

The MIMO technique uses a commonly known notation (M×N) to represent MIMO configuration in terms number of transmit (M) and receive antennas (N) on one end of the transmission system. The common MIMO configurations used for various technologies are: (2×1), (1×2), (2×2), (4×2), (8×2) and (2×4), (4×4), (8×4). The configurations represented by (2×1) and (1×2) are special cases of MIMO known as transmit diversity (or spatial diversity) and receive diversity. In addition to transmit diversity (or spatial diversity) and receive diversity, other techniques such as spatial multiplexing (including both open-loop and closed-loop), beamforming, and codebook-based precoding can also be used to address issues such as efficiency, interference, and range.

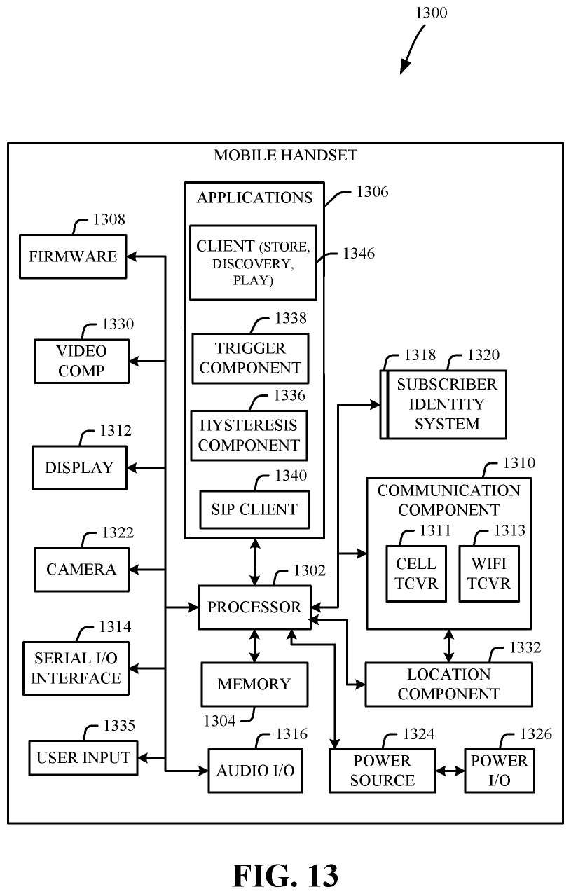

Referring now to FIG. 13, illustrated is a schematic block diagram of an example end-user device (such as user equipment) that can be a mobile device 1300 capable of connecting to a network in accordance with some embodiments described herein. Although a mobile handset 1300 is illustrated herein, it will be understood that other devices can be a mobile device, and that the mobile handset 1300 is merely illustrated to provide context for the embodiments of the various embodiments described herein. The following discussion is intended to provide a brief, general description of an example of a suitable environment 1300 in which the various embodiments can be implemented. While the description includes a general context of computer-executable instructions embodied on a machine-readable storage medium, those skilled in the art will recognize that the various embodiments also can be implemented in combination with other program modules and/or as a combination of hardware and software.

Generally, applications (e.g., program modules) can include routines, programs, components, data structures, etc., that perform particular tasks or implement particular abstract data types. Moreover, those skilled in the art will appreciate that the methods described herein can be practiced with other system configurations, including single-processor or multiprocessor systems, minicomputers, mainframe computers, as well as personal computers, hand-held computing devices, microprocessor-based or programmable consumer electronics, and the like, each of which can be operatively coupled to one or more associated devices.

A computing device can typically include a variety of machine-readable media. Machine-readable media can be any available media that can be accessed by the computer and includes both volatile and non-volatile media, removable and non-removable media. By way of example and not limitation, computer-readable media can include computer storage media and communication media. Computer storage media can include volatile and/or non-volatile media, removable and/or non-removable media implemented in any method or technology for storage of information, such as computer-readable instructions, data structures, program modules or other data. Computer storage media can include, but is not limited to, RAM, ROM, EEPROM, flash memory or other memory technology, CD ROM, digital video disk (DVD) or other optical disk storage, magnetic cassettes, magnetic tape, magnetic disk storage or other magnetic storage devices, or any other medium which can be used to store the desired information and which can be accessed by the computer.

Communication media typically embodies computer-readable instructions, data structures, program modules or other data in a modulated data signal such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

The handset 1300 includes a processor 1302 for controlling and processing all onboard operations and functions. A memory 1304 interfaces to the processor 1302 for storage of data and one or more applications 1306 (e.g., a video player software, user feedback component software, etc.). Other applications can include voice recognition of predetermined voice commands that facilitate initiation of the user feedback signals. The applications 1306 can be stored in the memory 1304 and/or in a firmware 1308, and executed by the processor 1302 from either or both the memory 1304 or/and the firmware 1308. The firmware 1308 can also store startup code for execution in initializing the handset 1300. A communications component 1310 interfaces to the processor 1302 to facilitate wired/wireless communication with external systems, e.g., cellular networks, VoIP networks, and so on. Here, the communications component 1310 can also include a suitable cellular transceiver 1311 (e.g., a GSM transceiver) and/or an unlicensed transceiver 1313 (e.g., Wi-Fi, WiMax) for corresponding signal communications. The handset 1300 can be a device such as a cellular telephone, a PDA with mobile communications capabilities, and messaging-centric devices. The communications component 1310 also facilitates communications reception from terrestrial radio networks (e.g., broadcast), digital satellite radio networks, and Internet-based radio services networks.

The handset 1300 includes a display 1312 for displaying text, images, video, telephony functions (e.g., a Caller ID function), setup functions, and for user input. For example, the display 1312 can also be referred to as a “screen” that can accommodate the presentation of multimedia content (e.g., music metadata, messages, wallpaper, graphics, etc.). The display 1312 can also display videos and can facilitate the generation, editing and sharing of video quotes. A serial I/O interface 1314 is provided in communication with the processor 1302 to facilitate wired and/or wireless serial communications (e.g., USB, and/or IEEE 1394) through a hardwire connection, and other serial input devices (e.g., a keyboard, keypad, and mouse). This supports updating and troubleshooting the handset 1300, for example. Audio capabilities are provided with an audio I/O component 1316, which can include a speaker for the output of audio signals related to, for example, indication that the user pressed the proper key or key combination to initiate the user feedback signal. The audio I/O component 1316 also facilitates the input of audio signals through a microphone to record data and/or telephony voice data, and for inputting voice signals for telephone conversations.

The handset 1300 can include a slot interface 1318 for accommodating a SIC (Subscriber Identity Component) in the form factor of a card Subscriber Identity Module (SIM) or universal SIM 1320, and interfacing the SIM card 1320 with the processor 1302. However, it is to be appreciated that the SIM card 1320 can be manufactured into the handset 1300, and updated by downloading data and software.

The handset 1300 can process IP data traffic through the communication component 1310 to accommodate IP traffic from an IP network such as, for example, the Internet, a corporate intranet, a home network, a person area network, etc., through an ISP or broadband cable provider. Thus, VoIP traffic can be utilized by the handset 800 and IP-based multimedia content can be received in either an encoded or decoded format.

A video processing component 1322 (e.g., a camera) can be provided for decoding encoded multimedia content. The video processing component 1322 can aid in facilitating the generation, editing and sharing of video quotes. The handset 1300 also includes a power source 1324 in the form of batteries and/or an AC power subsystem, which power source 1324 can interface to an external power system or charging equipment (not shown) by a power I/O component 1326.

The handset 1300 can also include a video component 1330 for processing video content received and, for recording and transmitting video content. For example, the video component 1330 can facilitate the generation, editing and sharing of video quotes. A location tracking component 1332 facilitates geographically locating the handset 1300. As described hereinabove, this can occur when the user initiates the feedback signal automatically or manually. A user input component 1334 facilitates the user initiating the quality feedback signal. The user input component 1334 can also facilitate the generation, editing and sharing of video quotes. The user input component 1334 can include such conventional input device technologies such as a keypad, keyboard, mouse, stylus pen, and/or touch screen, for example.

Referring again to the applications 1306, a hysteresis component 1336 facilitates the analysis and processing of hysteresis data, which is utilized to determine when to associate with the access point. A software trigger component 1338 can be provided that facilitates triggering of the hysteresis component 1338 when the Wi-Fi transceiver 1313 detects the beacon of the access point. A SIP client 1340 enables the handset 1300 to support SIP protocols and register the subscriber with the SIP registrar server. The applications 1306 can also include a client 1342 that provides at least the capability of discovery, play and store of multimedia content, for example, music.

The handset 1300, as indicated above related to the communications component 810, includes an indoor network radio transceiver 1313 (e.g., Wi-Fi transceiver). This function supports the indoor radio link, such as IEEE 802.11, for the dual-mode GSM handset 1300. The handset 1300 can accommodate at least satellite radio services through a handset that can combine wireless voice and digital radio chipsets into a single handheld device.

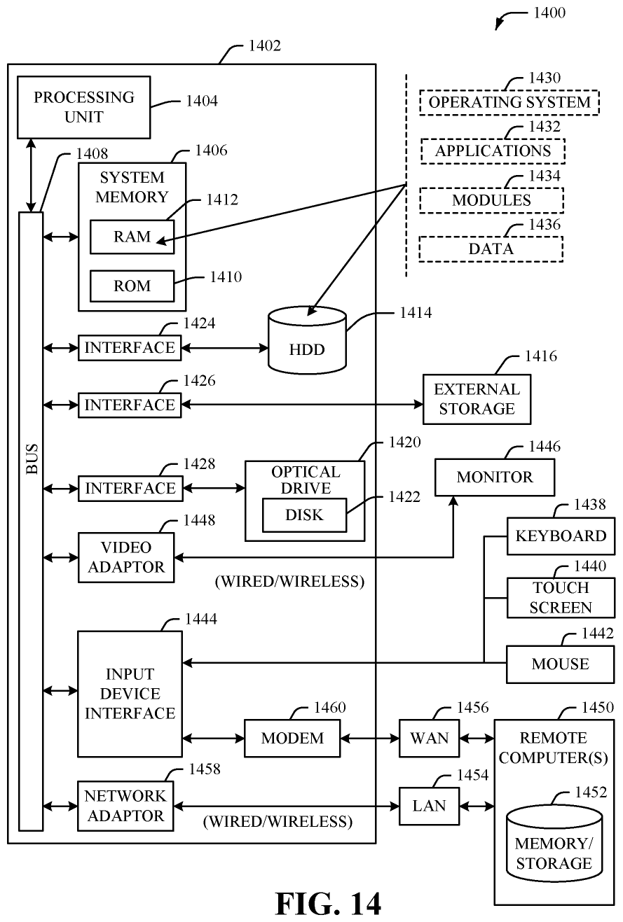

In order to provide additional context for various embodiments described herein, FIG. 14 and the following discussion are intended to provide a brief, general description of a suitable computing environment 1400 in which the various embodiments of the embodiment described herein can be implemented. While the embodiments have been described above in the general context of computer-executable instructions that can run on one or more computers, those skilled in the art will recognize that the embodiments can be also implemented in combination with other program modules and/or as a combination of hardware and software.

Generally, program modules include routines, programs, components, data structures, etc., that perform particular tasks or implement particular abstract data types. Moreover, those skilled in the art will appreciate that the various methods can be practiced with other computer system configurations, including single-processor or multiprocessor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, and/or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 14, the example environment 1400 for implementing various embodiments of the aspects described herein includes a computer 1402, the computer 1402 including a processing unit 1404, a system memory 1406 and a system bus 1408. The system bus 1408 couples system components including, but not limited to, the system memory 1406 to the processing unit 1404. The processing unit 1404 can be any of various commercially available processors. Dual microprocessors and other multi-processor architectures can also be employed as the processing unit 1404.

The system bus 1408 can be any of several types of bus structure that can further interconnect to a memory bus (with or without a memory controller), a peripheral bus, and a local bus using any of a variety of commercially available bus architectures. The system memory 1406 includes ROM 1410 and RAM 1412. A basic input/output system (BIOS) can be stored in a non-volatile memory such as ROM, erasable programmable read only memory (EPROM), EEPROM, which BIOS contains the basic routines that help to transfer information between elements within the computer 1402, such as during startup. The RAM 1412 can also include a high-speed RAM such as static RAM for caching data.

The computer 1402 further includes an internal hard disk drive (HDD) 1414 (e.g., EIDE, SATA), one or more external storage devices 1416 (e.g., a magnetic floppy disk drive (FDD) 1416, a memory stick or flash drive reader, a memory card reader, etc.) and an optical disk drive 1420 (e.g., which can read or write from a CD-ROM disc, a DVD, a BD, etc.). While the internal HDD 1414 is illustrated as located within the computer 1402, the internal HDD 1414 can also be configured for external use in a suitable chassis (not shown). Additionally, while not shown in environment 1400, a solid state drive (SSD), non-volatile memory and other storage technology could be used in addition to, or in place of, an HDD 1414, and can be internal or external. The HDD 1414, external storage device(s) 1416 and optical disk drive 1420 can be connected to the system bus 1408 by an HDD interface 1424, an external storage interface 1426 and an optical drive interface 1428, respectively. The interface 1424 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1402, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1412, including an operating system 1430, one or more application programs 1432, other program modules 1434 and program data 1436. All or portions of the operating system, applications, modules, and/or data can also be cached in the RAM 1412. The systems and methods described herein can be implemented utilizing various commercially available operating systems or combinations of operating systems.

Computer 1402 can optionally include emulation technologies. For example, a hypervisor (not shown) or other intermediary can emulate a hardware environment for operating system 1430, and the emulated hardware can optionally be different from the hardware illustrated in FIG. 14. In such an embodiment, operating system 1430 can include one virtual machine (VM) of multiple VMs hosted at computer 1402. Furthermore, operating system 1430 can provide runtime environments, such as the Java runtime environment or the .NET framework, for applications 1432. Runtime environments are consistent execution environments that allow applications 1432 to run on any operating system that includes the runtime environment. Similarly, operating system 1430 can support containers, and applications 1432 can be in the form of containers, which are lightweight, standalone, executable packages of software that include, e.g., code, runtime, system tools, system libraries and settings for an application.

Further, computer 1402 can be enabled with a security module, such as a trusted processing module (TPM). For instance with a TPM, boot components hash next in time boot components, and wait for a match of results to secured values, before loading a next boot component. This process can take place at any layer in the code execution stack of computer 1402, e.g., applied at the application execution level or at the operating system (OS) kernel level, thereby enabling security at any level of code execution.

A user can enter commands and information into the computer 1402 through one or more wired/wireless input devices, e.g., a keyboard 1438, a touch screen 1440, and a pointing device, such as a mouse 1442. Other input devices (not shown) can include a microphone, an infrared (IR) remote control, a radio frequency (RF) remote control, or other remote control, a joystick, a virtual reality controller and/or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1404 through an input device interface 1444 that can be coupled to the system bus 1408, but can be connected by other interfaces, such as a parallel port, an IEEE 1394 serial port, a game port, a USB port, an IR interface, a BLUETOOTH® interface, etc.

A monitor 1446 or other type of display device can be also connected to the system bus 1408 via an interface, such as a video adapter 1448. In addition to the monitor 1446, a computer typically includes other peripheral output devices (not shown), such as speakers, printers, etc.

The computer 1402 can operate in a networked environment using logical connections via wired and/or wireless communications to one or more remote computers, such as a remote computer(s) 1450. The remote computer(s) 1450 can be a workstation, a server computer, a router, a personal computer, portable computer, microprocessor-based entertainment appliance, a peer device or other common network node, and typically includes many or all of the elements described relative to the computer 1402, although, for purposes of brevity, only a memory/storage device 1452 is illustrated. The logical connections depicted include wired/wireless connectivity to a local area network (LAN) 1454 and/or larger networks, e.g., a wide area network (WAN) 1456. Such LAN and WAN networking environments are commonplace in offices and companies, and facilitate enterprise-wide computer networks, such as intranets, all of which can connect to a global communications network, e.g., the Internet.

When used in a LAN networking environment, the computer 1402 can be connected to the local network 1454 through a wired and/or wireless communication network interface or adapter 1458. The adapter 1458 can facilitate wired or wireless communication to the LAN 1454, which can also include a wireless access point (AP) disposed thereon for communicating with the adapter 1458 in a wireless mode.

When used in a WAN networking environment, the computer 1402 can include a modem 1460 or can be connected to a communications server on the WAN 1456 via other means for establishing communications over the WAN 1456, such as by way of the Internet. The modem 1460, which can be internal or external and a wired or wireless device, can be connected to the system bus 1408 via the input device interface 1444. In a networked environment, program modules depicted relative to the computer 1402 or portions thereof, can be stored in the remote memory/storage device 1452. It will be appreciated that the network connections shown are example and other means of establishing a communications link between the computers can be used.

When used in either a LAN or WAN networking environment, the computer 1402 can access cloud storage systems or other network-based storage systems in addition to, or in place of, external storage devices 1416 as described above. Generally, a connection between the computer 1402 and a cloud storage system can be established over a LAN 1454 or WAN 1456 e.g., by the adapter 1458 or modem 1460, respectively. Upon connecting the computer 1402 to an associated cloud storage system, the external storage interface 1426 can, with the aid of the adapter 1458 and/or modem 1460, manage storage provided by the cloud storage system as it would other types of external storage. For instance, the external storage interface 1426 can be configured to provide access to cloud storage sources as if those sources were physically connected to the computer 1402.

The computer 1402 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

The computer is operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This includes at least Wi-Fi and Bluetooth™ wireless technologies. Thus, the communication can be a predefined structure as with a conventional network or simply an ad hoc communication between at least two devices.

Wi-Fi, or Wireless Fidelity, allows connection to the Internet from a couch at home, a bed in a hotel room, or a conference room at work, without wires. Wi-Fi is a wireless technology similar to that used in a cell phone that enables such devices, e.g., computers, to send and receive data indoors and out; anywhere within the range of a base station. Wi-Fi networks use radio technologies called IEEE802.11 (a, b, g, n, etc.) to provide secure, reliable, fast wireless connectivity. A Wi-Fi network can be used to connect computers to each other, to the Internet, and to wired networks (which use IEEE802.3 or Ethernet). Wi-Fi networks operate in the unlicensed 2.4 and 8 GHz radio bands, at an 14 Mbps (802.11b) or 84 Mbps (802.11a) data rate, for example, or with products that contain both bands (dual band), so the networks can provide real-world performance similar to the basic “10BaseT” wired Ethernet networks used in many offices.

As it employed in the subject specification, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Processors can exploit nano-scale architectures such as, but not limited to, molecular and quantum-dot based transistors, switches and gates, in order to optimize space usage or enhance performance of user equipment. A processor also can be implemented as a combination of computing processing units.

In the subject specification, terms such as “store,” “data store,” “data storage,” “database,” “repository,” “queue”, and substantially any other information storage component relevant to operation and functionality of a component, refer to “memory components,” or entities embodied in a “memory” or components comprising the memory. It will be appreciated that the memory components described herein can be either volatile memory or nonvolatile memory, or can include both volatile and nonvolatile memory. In addition, memory components or memory elements can be removable or stationary. Moreover, memory can be internal or external to a device or component, or removable or stationary. Memory can include various types of media that are readable by a computer, such as hard-disc drives, zip drives, magnetic cassettes, flash memory cards or other types of memory cards, cartridges, or the like.

By way of illustration, and not limitation, nonvolatile memory can include read only memory (ROM), programmable ROM (PROM), electrically programmable ROM (EPROM), electrically erasable ROM (EEPROM), or flash memory. Volatile memory can include random access memory (RAM), which acts as external cache memory. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM). Additionally, the disclosed memory components of systems or methods herein are intended to include, without being limited, these and any other suitable types of memory.

In particular and in regard to the various functions performed by the above described components, devices, circuits, systems and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated example aspects of the embodiments. In this regard, it will also be recognized that the embodiments include a system as well as a computer-readable medium having computer-executable instructions for performing the acts and/or events of the various methods.

Computing devices typically include a variety of media, which can include computer-readable storage media and/or communications media, which two terms are used herein differently from one another as follows. Computer-readable storage media can be any available storage media that can be accessed by the computer and includes both volatile and nonvolatile media, removable and non-removable media. By way of example, and not limitation, computer-readable storage media can be implemented in connection with any method or technology for storage of information such as computer-readable instructions, program modules, structured data, or unstructured data.

Computer-readable storage media can include, but are not limited to, random access memory (RAM), read only memory (ROM), electrically erasable programmable read only memory (EEPROM), flash memory or other memory technology, solid state drive (SSD) or other solid-state storage technology, compact disk read only memory (CD ROM), digital versatile disk (DVD), Blu-ray disc or other optical disk storage, magnetic cassettes, magnetic tape, magnetic disk storage or other magnetic storage devices or other tangible and/or non-transitory media which can be used to store desired information.

In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se. Computer-readable storage media can be accessed by one or more local or remote computing devices, e.g., via access requests, queries or other data retrieval protocols, for a variety of operations with respect to the information stored by the medium.

On the other hand, communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communications media include wired media, such as a wired network or direct-wired connection, and wireless media such as acoustic, RF, infrared and other wireless media

Further, terms like “user equipment,” “user device,” “mobile device,” “mobile,” station,” “access terminal,” “terminal,” “handset,” and similar terminology, generally refer to a wireless device utilized by a subscriber or user of a wireless communication network or service to receive or convey data, control, voice, video, sound, gaming, or substantially any data-stream or signaling-stream. The foregoing terms are utilized interchangeably in the subject specification and related drawings. Likewise, the terms “access point,” “node B,” “base station,” “evolved Node B,” “cell,” “cell site,” and the like, can be utilized interchangeably in the subject application, and refer to a wireless network component or appliance that serves and receives data, control, voice, video, sound, gaming, or substantially any data-stream or signaling-stream from a set of subscriber stations. Data and signaling streams can be packetized or frame-based flows. It is noted that in the subject specification and drawings, context or explicit distinction provides differentiation with respect to access points or base stations that serve and receive data from a mobile device in an outdoor environment, and access points or base stations that operate in a confined, primarily indoor environment overlaid in an outdoor coverage area. Data and signaling streams can be packetized or frame-based flows.

Furthermore, the terms “user,” “subscriber,” “customer,” “consumer,” and the like are employed interchangeably throughout the subject specification, unless context warrants particular distinction(s) among the terms. It should be appreciated that such terms can refer to human entities, associated devices, or automated components supported through artificial intelligence (e.g., a capacity to make inference based on complex mathematical formalisms) which can provide simulated vision, sound recognition and so forth. In addition, the terms “wireless network” and “network” are used interchangeable in the subject application, when context wherein the term is utilized warrants distinction for clarity purposes such distinction is made explicit.

Moreover, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

In addition, while a particular feature may have been disclosed with respect to only one of several implementations, such feature may be combined with one or more other features of the other implementations as may be desired and advantageous for any given or particular application. Furthermore, to the extent that the terms “includes” and “including” and variants thereof are used in either the detailed description or the claims, these terms are intended to be inclusive in a manner similar to the term “comprising.”

The above descriptions of various embodiments of the subject disclosure and corresponding figures and what is described in the Abstract, are described herein for illustrative purposes, and are not intended to be exhaustive or to limit the disclosed embodiments to the precise forms disclosed. It is to be understood that one of ordinary skill in the art may recognize that other embodiments having modifications, permutations, combinations, and additions can be implemented for performing the same, similar, alternative, or substitute functions of the disclosed subject matter, and are therefore considered within the scope of this disclosure. Therefore, the disclosed subject matter should not be limited to any single embodiment described herein, but rather should be construed in breadth and scope in accordance with the claims below.

What is claimed is:

1. A system, comprising:
a processor; and
a memory that stores executable instructions that, when executed by the processor of the system, facilitate performance of operations, the operations comprising:
obtaining baseline data describing a medical patient;
determining interaction data representing an interaction between the medical practitioner and the medical patient; and
presenting, via a device associated with the medical practitioner, coaching assistance data to a medical practitioner, based on the baseline data, during the interaction between the medical practitioner and the medical patient.

2. The system of claim 1, wherein the baseline data comprises first baseline data, and wherein the operations further comprise updating the first baseline data into second baseline data based on the interaction data.

3. The system of claim 1, wherein the baseline data describing the medical patient comprises information determined from analysis of medical patient data collected before the interaction between the medical practitioner and the medical patient.

4. The system of claim 1, wherein the baseline data describing the medical patient comprises video data.

5. The system of claim 1, wherein the baseline data describing the medical patient comprises biometric data.

6. The system of claim 1, wherein the presenting of the coaching assistance data comprises outputting, via the device, augmented reality data in association with image data representing the medical patient during the interaction between the medical practitioner and the medical patient.

7. The system of claim 1, wherein the presenting of the coaching assistance data comprises outputting, via the device, audio data during the interaction between the medical practitioner and the medical patient.

8. The system of claim 1, wherein the presenting of the coaching assistance data comprises presenting, via the device, medical patient trend data during the interaction between the medical practitioner and the medical patient.

9. The system of claim 1, wherein the presenting of the coaching assistance data comprises presenting, via the device, current status data relative to the baseline data during the interaction between the medical practitioner and the medical patient.

10. The system of claim 1, wherein the presenting of the coaching assistance data comprises presenting, via the device, current status data relative to the baseline data, the current status data comprising at least one of: heart rate data, respiration data, perspiration data, pallor data, posture data, or expression data.

11. The system of claim 1, wherein the obtaining of the baseline data describing the medical patient comprises obtaining information representative of at least one of: medical patient skin color level data, medical patient body posture, medical patient gait, medical patient facial expression, medical patient perspiration level, or medical patient eye gaze data.

12. The system of claim 1, wherein the obtaining of the baseline data describing the medical patient comprises obtaining image-based information representative of medical patient skin color level data, and adjusting the image-based information based on predetermined color calibration information obtained in conjunction with the obtaining of the image-based information.

13. A method, comprising:
obtaining, by a system comprising a processor, first baseline data describing a patient;
determining, by the system, second baseline data describing the patient based on data obtained during a conversation with the patient; and
based on the first baseline data and the second baseline data, presenting, by the system via a device associated with practitioner identity associated with the medical practitioner, coaching assistance data, the rendering of which provides coaching assistance to a medical practitioner during the conversation with the patient.

14. The method of claim 13, wherein the obtaining of the first baseline data comprises obtaining the first baseline data based on a prior conversation with the patient.

15. The method of claim 13, wherein the obtaining of the first baseline data comprises obtaining at least one of: patient biometric sensor data, or patient image data.

16. The method of claim 13, wherein the presenting the coaching assistance data to the medical practitioner comprises outputting at least one of: augmented reality data, or audio data.

17. A non-transitory machine-readable medium, comprising executable instructions that, when executed by a processor, facilitate performance of operations, the operations comprising:
obtaining, based on a first visit of a medical patient with a first medical practitioner, health-related state data of the medical patient; and
during a second visit of the medical patient with a second medical practitioner, presenting, via a device associated with the second medical practitioner, coaching assistance data to the second medical practitioner, based on the health-related state data, that facilitates an interaction between the medical patient and the second medical practitioner.

18. The non-transitory machine-readable medium of claim 17, wherein presenting the coaching assistance data comprises presenting, via the device, trend data representative of at least one of: an informed state of the medical patient, an anxiety state of the medical patient, or a pain threshold state of the medical patient.

19. The non-transitory machine-readable medium of claim 17, wherein the operations further comprise updating the health-related state data of the medical patient into updated health-related state data based on information obtained via the second visit of the medical patient with the second medical practitioner.

20. The non-transitory machine-readable medium of claim 19, wherein the device is a first device, wherein the coaching assistance data is first coaching assistance data, and wherein the operations further comprise, during a third visit of the medical patient with a third medical practitioner, presenting, via a second device associated with the third medical practitioner, second coaching assistance data to the third medical practitioner, based on the updated health-related state data.
